(19) United States
(12) Patent Application Publication
(10) Pub. No.: US 2026/0327163 A1
(43) Pub. Date: Oct. 22, 2026

(54) SYSTEMS AND METHODS FOR PRODUCING LINEAR OLEFINS UTILIZING A SUPERCRITICAL WATER UNIT

(71) Applicant: Saudi Arabian Oil Company, Dhahran (SA)

(72) Inventors: Ki-Hyouk Choi, Dhahran (SA); Young-Kyoung Ahn, Dhahran (SA); Joo-Hyeong Lee, Dhahran (SA); Mohammed Rashid Aldossary, Dhahran (SA)

(73) Assignee: Saudi Arabian Oil Company, Dhahran (SA)

(21) Appl. No.: 19/180,416

(22) Filed: Apr. 16, 2025

(51) Int. Cl. C07C 4/04; B01D 3/14; B01D 15/20; B01D 15/24; B01J 3/00; B01J 19/24; C07C 7/04; C07C 7/12; C10G 67/04

(52) U.S. Cl. CPC C07C 4/04; B01D 3/143; B01D 15/203; B01D 15/245; B01J 3/008; B01J 19/245; C07C 7/04; C07C 7/12; C10G 67/049; C10G 2300/1003; C10G 2300/1077; C10G 2300/202; C10G 2300/301; C10G 2400/22

(57) ABSTRACT

A process for producing linear alpha olefins utilizing a supercritical water unit may comprise introducing a hydrocarbon-containing stream and water to a supercritical water unit, wherein the water is either at supercritical conditions prior to entering the supercritical water unit or converted to supercritical conditions within the supercritical water unit; and upgrading the hydrocarbon-containing stream with the water within the supercritical water unit, thereby forming a linear alpha olefin-containing product, and wherein: the hydrocarbon-containing stream comprises greater than or equal to 90 vol. % paraffins, the hydrocarbon-containing stream comprises from 0.1 vol. % to 10 vol. % aromatics, and the hydrocarbon-containing stream comprises from 0.1 ppm to 100 ppm sulfur.

### FIELD

The present disclosure relates to the field of hydrocarbon upgrading. More particularly, it relates to systems and methods of upgrading hydrocarbons to linear alpha olefins (LAO) utilizing a supercritical water unit, as detailed herein.

### BACKGROUND

Olefins, including alpha-olefins, are useful and valuable chemicals when used as a raw material. For example, alpha olefins can be used as a raw material for the production of low density polyethylene (LDPE), linear low density polyethylene (LLDPE), high density polyethylene (HDPE), polyalphaolefin (PAO), linear alkyl benzene (LAB), and linear alkyl benzene sulfonate (LABS).

Alpha-olefins for use as a raw material are generally produced as the primary production product, such as in the Ziegler process, which utilizes organometallic catalyst and oligomerization of light olefins or in catalytic dehydrogenation of n-Paraffins, such as in UOP's Pacol process. However, catalytic processes are very expensive and utilize limited feedstocks such as ethylene.

Non-catalytic processes for wider ranges of feedstocks include thermal cracking of residue or crude oils. Thermal cracking methods generally subject C₂₀-C₃₀ range hydrocarbons, including waxes, to elevated temperatures in the presence of steam, which results in cracking. Conversion in thermal cracking processes is about 25% and the product is 90-95% alpha olefins. However, thermal cracking products from wax as a feed contain a wide range alpha olefins, only some of which are useful in polyalphaolefin production. For crude oils feeds, desired alpha olefin fractions are not readily or economically separable from other components such as n-paraffins and aromatics, such that further thermal decomposition of the alpha olefin compounds results in formation of aromatics through cyclization and alpha olefins compounds' inherent instability.

Accordingly, methods are desired by which alpha olefins, and particularly linear alpha olefins, may be selectively produced from a wider range of feedstocks. Supercritical water is one such pathway that has been investigated to produce alpha-olefins from various feedstocks. For example, supercritical water is known to have unique properties for upgrading, desulfurization, and demetallization of petroleum-based feedstock, among other processes.

### SUMMARY

When hydrocarbons are exposed to supercritical water, alkyl radicals formed under thermal decomposition, including alkyl aromatic radicals, can undergo propagation by two paths: hydrogen abstraction and beta-scission. These propagation pathways are moderated by the supercritical water acting as a diluent, creating a “cage effect” that surrounds local groups of radicals and suppresses inter-radical and intermolecular reactions.

Hydrogen abstraction takes hydrogen from other compounds, with the alkyl radicals subsequently being converted to alkanes. However, hydrogen abstraction reactions are suppressed in supercritical water reactions due to the dilution effect, making it difficult to find hydrogen donor compounds and thereby only a minor amount of alpha olefin production occurs by this pathway. Positively however, this dilution effect also suppresses isomerization from alpha olefins to inner olefins (where the double bond is not at the alpha position).

In beta-scission, the alkyl radical cracks to produce an alkyl radical and alpha-olefins. Beta-scission does not require additional molecules. Accordingly, due to not requiring donor compounds, beta-scission of alkyl radicals is increased under supercritical water conditions. Thus, under supercritical water conditions, more alpha olefins tend to be formed than under conventional thermal cracking conditions, primarily under the beta scission pathway.

However, the favoring of beta-scission over hydrogen abstraction is not itself completely beneficial to alpha olefin yield. Particularly, due to the dilution effect and a lack of hydrogen donors, radical-mediated cyclization of alpha olefins to aromatics and/or may occur, again resulting in depressed alpha olefin yields.

Consequently, while supercritical water upgrading exhibits potential for conversion of hydrocarbon fractions, further research is needed on how to balance the various reaction pathways in supercritical water to best increase the yield and subsequent separation of alpha olefins, and particularly linear alpha olefins without risking conversion to aromatic form.

Accordingly, embodiments herein balance the various pathways by providing specific sources of hydrogen donor compounds with tailored hydrocarbon-containing streams, such that undesired isomerization and cyclization are balanced and linear alpha olefin yield is improved.

Particularly, in at least one embodiment, a hydrocarbon-containing stream comprising less from 0.1 ppm to 100 ppm sulfur and greater than or equal to 90 vol % paraffins is provided to a supercritical water unit. The supercritical water operates to first form alkyl radicals and alpha olefins via the beta scission pathway. Concurrently, cleavage of carbon-sulfur bonds (such as but not limited to aliphatic chains) results in the formation of hydrogen sulfide, forming sulfanyl and hydrogen radicals which then act as hydrogen donors to encourage hydrogen abstraction and suppress cyclization of the alkyl radicals and alpha olefins to aromatics. This may have increased benefit especially where the non-paraffinic fraction comprises aromatics, which may otherwise be prone to form coke through condensation reactions without adequate hydrogen abstraction. The net result may be an increase in the linear alpha olefin yield of the supercritical water unit.

Further, the balancing of sulfur content in the feed enables subsequent separation of the linear alpha olefins from the remainder of the feed. Particularly, subsequent hydrodesulfurization of the alpha olefins may not be desired due to the potential for saturation and reversion of the alpha olefins to paraffins. Conversely, while adsorption may be used to separate the alpha olefins, excess sulfur in the feed may result in competition for adsorption sites between sulfur-having hydrocarbons and olefins, such that alpha olefin recovery and purity is lacking. However, as supercritical water itself has a desulfurization function, by tailoring the hydrocarbon-containing stream to between 0.1 ppm to 100 ppm sulfur, sulfur participation in the radical-mediated reactions of the supercritical water unit can be maximized, such that later competition for adsorption sites is minimized.

As previously stated, embodiments herein may balance the various supercritical water upgrading pathways by providing specific sources of hydrogen donor compounds with tailored hydrocarbon-containing streams. Accordingly, in at least one other embodiment herein, a waste plastics fraction containing polyolefins may be provided with the hydrocarbon-containing stream to the supercritical water unit. The waste plastics fraction, as having a high hydrogen content, may act as an additional hydrogen donor, on top of having the added benefit of forming alpha olefins as part of the initial radical reactions. Further yet, the waste plastics fraction may be mixed with an aromatics stream to pre-swell the waste plastics, increasing distribution of hydrogen donors within the supercritical medium (and to hydrogen donor receptors such as aromatics) within the non-paraffinic fraction of the hydrocarbon-containing stream. Finally, mixing of the swelled waste plastics with the hydrocarbon-containing stream prior to introduction to the water may suppress subsequent hydrolysis reactions with near-critical water. These hydrolysis reactions would otherwise yield oxygenates that would be more inert in the SCW conditions, again reducing desired yields.

In accordance with one embodiment herein, a process for producing linear alpha olefins utilizing a supercritical water unit may comprise introducing a hydrocarbon-containing stream and water to a supercritical water unit, wherein the water is either at supercritical conditions prior to entering the supercritical water unit or converted to supercritical conditions within the supercritical water unit; and upgrading the hydrocarbon-containing stream with the water within the supercritical water unit, thereby forming a linear alpha olefin-containing product, and wherein: the hydrocarbon-containing stream comprises greater than or equal to 90 vol. % paraffins, the hydrocarbon-containing stream comprises from 0.1 vol. % to 10 vol. % aromatics, and the hydrocarbon-containing stream comprises from 0.1 ppm to 100 ppm sulfur.

In accordance with another embodiment herein, a system for producing linear alpha olefins may comprise a pre-treatment unit, a supercritical water unit, one or more separation units, a distillation column, an adsorption unit, a first mixing unit, and a second mixing unit.

In at least the previous embodiment, the supercritical water unit may be fluidly connected downstream from the pre-treatment unit; the one or more separation units may be fluidly connected downstream of the supercritical water unit; the distillation column may be fluidly connected downstream of the supercritical water unit and the one or more separation units; the adsorption unit may be fluidly connected downstream of the supercritical water unit; the first mixing unit may be fluidly connected downstream of the adsorption unit; the second mixing unit may be fluidly connected downstream of the first mixing unit and the pre-treatment unit; and the second mixing unit may be fluidly connected upstream from the supercritical water unit.

In at least the previous embodiment, the pre-treatment unit may be configured to receive a carbon-containing stream; the supercritical water unit may be configured to receive water and a hydrocarbon-containing stream-swollen waste plastic stream mixture from the second mixing unit; the supercritical water unit may be configured to operate under supercritical water conditions; the one or more separation units may be configured to receive a linear alpha olefin-containing product from the supercritical water unit; the distillation column may be configured to receive a liquid distillate product or a liquid hydrocarbon stream from the one or more separation units; the adsorption unit may be configured to receive a middle distillate, a heavy distillate, or both from the distillation column; the adsorption unit may be configured to produce at least a linear alpha olefin product; the first mixing unit may be configured to receive an unadsorbed aromatics fraction from the adsorption unit and a waste plastics stream; and the second mixing unit may be configured to receive a hydrocarbon-containing stream from the pre-treatment unit and a swollen waste plastics stream from the first mixing unit.

Additional features and advantages of the described embodiments will be set forth in the detailed description which follows, and in part will be readily apparent to those skilled in the art from that description or recognized by practicing the described embodiments, including the detailed description which follows as well as the claims.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 illustrates a system for producing linear alpha olefins utilizing a supercritical water unit, according to embodiments herein;

FIG. 2 illustrates a simplified version of the system of FIG. 1, according to embodiments herein;

FIG. 3 illustrates another simplified version of the system of FIG. 1 with the additional provision of an upstream pretreatment unit comprising a hydroprocessor, according to embodiments herein;

FIG. 4 illustrates another simplified version of the system of FIG. 1 with the additional provision of an upstream pretreatment unit comprising a hydroprocessor and a solvent deasphalting unit, according to embodiments herein;

FIG. 5 illustrates another simplified version of the system of FIG. 1 with the additional provision of a waste plastics fraction to the supercritical water unit, according to embodiments herein;

FIG. 6 illustrates another simplified version of the system of FIG. 1 with the additional provision of a first mixer for the swelling of a waste plastics fraction with an aromatics stream and a second mixer for the mixing of the hydrocarbon-containing stream and the swollen waste plastics fraction, according to embodiments herein; and

For the purpose of describing the simplified schematic illustrations and descriptions of the relevant figures, the numerous valves, temperature sensors, electronic controllers and the like that may be employed and well known to those of ordinary skill in the art of certain chemical processing operations are not included. It should be understood that these components are within the spirit and scope of the present embodiments disclosed. However, operational components, such as those described in the present disclosure, may be added to the embodiments described in this disclosure.

It should further be noted that arrows in the drawings refer to process streams. However, the arrows may equivalently refer to transfer lines, which may serve to transfer process streams between two or more system components. Additionally, arrows that connect to system components define inlets or outlets in each given system component. The arrow direction corresponds generally with the major direction of movement of the materials of the stream contained within the physical transfer line signified by the arrow. Furthermore, arrows, which do not connect two or more system components, signify a product stream, which exits the depicted system, or a system inlet stream, which enters the depicted system. Product streams may be further processed in accompanying chemical processing systems or may be commercialized as end products. System inlet streams may be streams transferred from accompanying chemical processing systems or may be non-processed feedstock streams. Some arrows may represent recycle streams, which are effluent streams of system components that are recycled back into the system. However, it should be understood that any represented recycle stream, in some embodiments, may be replaced by a system inlet stream of the same material, and that a portion of a recycle stream may exit the system as a product.

Additionally, arrows in the drawings may schematically depict process steps of transporting a stream from one system component to another system component. For example, an arrow from one system component pointing to another system component may represent “passing” a system component effluent to another system component, which may include the contents of a process stream “exiting” or being “removed” from one system component and “introducing” the contents of that product stream to another system component.

It should be understood that according to the embodiments presented in the relevant figures, an arrow between two system components may signify that the stream is not processed between the two system components. In other embodiments, the stream signified by the arrow may have substantially the same composition throughout its transport between the two system components. Additionally, it should be understood that in embodiments, an arrow may represent that at least 75 wt. %, at least 90 wt. %, at least 95 wt. %, at least 99 wt. %, at least 99.9 wt. %, or even 100 wt. % of the stream is transported between the system components. As such, in embodiments, less than all of the stream signified by an arrow may be transported between the system components, such as if a slip stream is present.

It should be understood that two or more process streams are “mixed” or “combined” when two or more lines intersect in the schematic flow diagrams of the relevant figures. Mixing or combining may also include mixing by directly introducing both streams into a like reactor, separation unit, or other system component. For example, it should be understood that when two streams are depicted as being combined directly prior to entering a separation unit or reactor, that in embodiments the streams could equivalently be introduced into the separation unit or reactor and be mixed in the reactor. Alternatively, when two streams are depicted to independently enter a system component, they may in embodiments be mixed together before entering that system component.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

As previously stated, embodiments described herein generally relate to the field of hydrocarbon upgrading, and particularly, to systems and methods of upgrading hydrocarbons to linear alpha olefins utilizing a supercritical water unit.

As used in this disclosure, a “separation unit” refers to any separation device that at least partially separates one or more chemicals that are mixed in a process stream from one another. For example, a separation unit may selectively separate differing chemical species, phases, or sized material from one another, forming one or more chemical fractions. Examples of separation units include, without limitation, distillation columns, flash drums, knock-out drums, knock-out pots, centrifuges, cyclones, filtration devices, traps, scrubbers, expansion devices, membranes, solvent extraction devices, and the like. It should be understood that separation processes described in this disclosure may not completely separate all of one chemical constituent from all of another chemical constituent. It should be understood that the separation processes described in this disclosure “at least partially” separate different chemical components from one another, and that even if not explicitly stated, it should be understood that separation may include only partial separation. As used in this disclosure, one or more chemical constituents may be “separated” from a process stream to form a new process stream. Generally, a process stream may enter a separation unit and be divided, or separated, into two or more process streams of desired composition. It should be understood that an “effluent” generally refers to a stream that exits a system component such as a separation unit, a reactor, or reaction zone, following a particular reaction or separation, and generally has a different composition (at least proportionally) than the stream that entered the separation unit, reactor, or reaction zone.

It should further be understood that streams and/or fractions, as the terms are used interchangeably herein, may be named for the components of the stream, and the component for which the stream is named may be the major component of the stream (such as comprising from 50 weight percent (wt. %), from 70 wt. %, from 90 wt. %, from 95 wt. %, from 99 wt. %, from 99.5 wt. %, or even from 99.9 wt. % of the contents of the stream to 100 wt. % of the contents of the stream). It should also be understood that components of a stream are disclosed as passing from one system component to another when a stream comprising that component is disclosed as passing from that system component to another. For example, a disclosed “aromatics stream” passing from a first system component to a second system component should be understood to equivalently disclose “aromatics” passing from a first system component to a second system component, and the like.

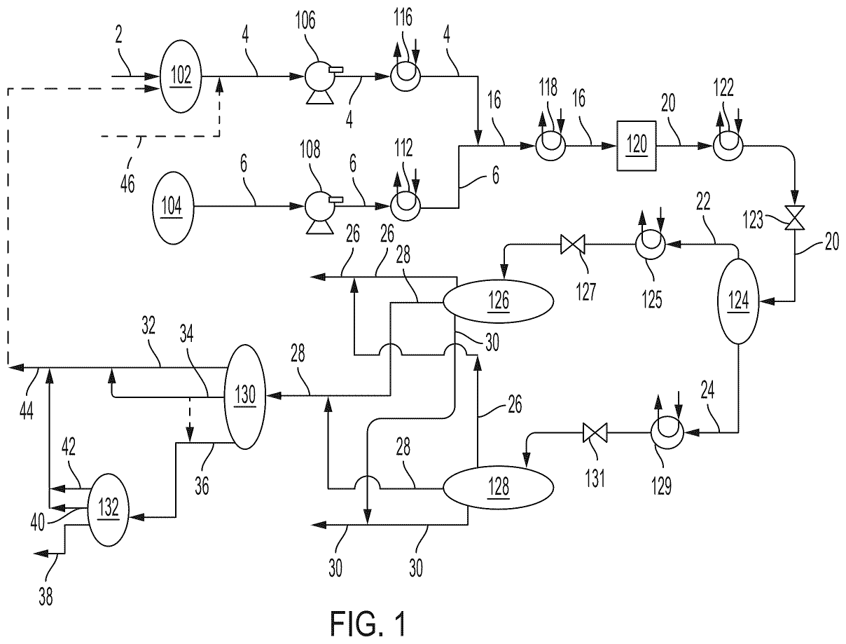

Referring initially to FIG. 1, illustrated is a system 100 for upgrading a hydrocarbon-containing stream 4 in a supercritical water unit 120 with water 6. As shown in FIG. 1, the water 6 may originate from a water storage medium 104, which may be fluidly connected upstream of the supercritical water unit 120. The hydrocarbon-containing stream 4 may originate from a pretreatment unit 102, which may be fluidly connected upstream of the supercritical water unit 120.

The pretreatment unit 102 may itself comprise one more units capable of conducting operations on a carbon-containing stream 2, the operations including, but not limited to, hydrogenation, hydrodesulfurization, hydrodenitrogenation, hydrodemetallization, hydrocracking, hydrotreatment, thermal cracking, isomerization, alkylation, dealkylation, condensation, aromatization, dehydrogenation, physical fractionation, distillation, solvent extraction, solvent deasphalting, delayed coking, crushing, screening, conveying, extruding, etc. Accordingly, the pretreatment unit 102 may pretreat the carbon-containing stream 2 to form the hydrocarbon-containing stream 4. The carbon-containing stream 2 may itself comprise any carbon-containing product (either solid, liquid, or gas) that may be converted to a hydrocarbon form, and may be contained in one or more storage mediums (not illustrated). For example, in one non-limiting embodiment where the pretreatment unit 102 comprise a hydroprocessor, the carbon-containing stream 2 may comprise a vacuum gas oil, a vacuum residue fraction, or combinations thereof. Alternatively or additionally, the carbon-containing stream 2 may comprise a crude oil, coal, biomass, plastics, carbon monoxide/carbon dioxide (for conversion to syngas, alcohols, and/or hydrocarbons), as well as refined or derived variations of the previous.

The pretreatment unit 102 may include, but may not be limited to, a hydroprocessing unit selected from a hydrocracker, a hydrotreater, or both; a thermal cracker; a solvent extraction unit (such as a solvent deasphalter); a Fischer-Tropsch synthesis unit; a hydrogenation unit (such as a syngas unit); or combinations thereof.

For example, in at least one embodiment, the pretreatment unit 102 may comprise a hydroprocessor and the hydrocarbon-containing stream 4 may comprise a hydroprocessor bottoms residue fraction, a hydroprocessor unconverted oil fraction, or both. Without being limited by theory, sourcing the hydrocarbon-containing stream 4 from the bottoms residue or unconverted oil fraction of a hydroprocessor may have the advantage of limiting overabundance of sulfur, nitrogen, and organometallics/heteroatoms in the feed to the supercritical upgrading unit. Thereby, the hydrocarbon-containing stream 4 may have a balanced feed which may maximize the presence of sulfur radicals in the reactions to produce linear alpha olefins (LAO) while also reducing the presence of sulfur-hydrocarbon species in the subsequent separation of linear alpha olefins by adsorption. Additionally or alternatively, a solvent extraction unit, such as a solvent deasphalter, may be fluidly connected upstream of the hydroprocessor as part of the pretreatment unit 102 to remove additional undesired species from the carbon-containing stream 2 prior to forming the hydrocarbon-containing stream 4.

As previously stated, the pretreatment unit 102 may be configured to pretreat the carbon-containing stream 2 to form the hydrocarbon-containing stream 4. In at least some embodiments, the pretreatment may form a hydrocarbon-containing stream 4 having greater than or equal to 90 vol. % paraffins (such as from 90 vol. % to 99.9 vol. %, from 0.1 vol. % to 10 vol. % aromatics, from 0.1 ppm to 100 ppm sulfur, or combinations thereof. Parts per million sulfur, as used herein, refers to both the relative content of elemental sulfur as well as the amount of sulfur-containing compounds (heteroatoms, aliphatic sulfur chains, etc.) within the stream. As explained in greater detail below, the balancing of the percent of paraffins, aromatics, and sulfur in the hydrocarbon-containing stream 4 may result in increased linear alpha olefin yields as well as decreased competition for adsorption sites when the linear alpha olefins are later separated.

Owing to the various functions possible for the pretreatment unit 102, the carbon-containing stream 2 may also be treated in the pretreatment unit 102 such that the hydrocarbon-containing stream 4 has less than 10 ppm nitrogen (such as from 10 ppm to 0 ppm); less than 100 ppm oxygen (such as from 100 ppm to 0 ppm oxygen); a T₉₅ boiling point of from 174° C. to 600° C. (such as from 280° C. to 565° C.); over 95 vol. % C₁₀-C₄₆ fractions (such as from 95 vol. % to 100 vol. %, and/or over 95 vol % C₁₆-C₄₆ fractions); or combinations thereof. As used herein, “T₉₅ boiling point” and/or “Tx boiling point” refers to a given stream having at least 95 wt. % fractions therein, or X wt. % fractions therein, having a boiling point within the given range. Accordingly, as used previously, the hydrocarbon-containing stream 4 may have at least 95 wt. % fractions having a boiling point of from 174° C. to 600° C. Similarly, “C₁₀-C₄₆ fractions” and/or “Cₓ-Cₓ₊ₙ fractions refers to a given stream having hydrocarbons with anywhere from 10 carbons (x carbons) to 46 carbons (x+n carbons), or any smaller range therein, such as from 10 carbons to 12 carbons, from 12 carbons to 20 carbons, etc.

As previously stated, the water 6 may originate from a water storage medium 104. However, the water 6 may also be pretreated such that it meets one or more criteria for supercritical water. For example, and in embodiments, the water 6 may comprise a demineralized water; a conductivity of less than 1 microsiemens per centimeter (μS/cm) (such as from 1 to 0.01 μS/cm; a sodium content of less than 5 micrograms per liter (μg/L) (such as from 0.001 μg/L to 5 μg/L); a chloride content of less than 5 micrograms per liter (such as from 0.001 μg/L to 5 μg/L); a silica content of less than 3 micrograms per liter (such as from 0.001 μg/L to 3 μg/L); or combinations thereof.

As previously stated, the system 100 may comprise the supercritical water unit 120. The supercritical water unit 120 is not particularly limited, and may be a tubular type reactor, a vessel-type reactor, or a combination tubular-vessel-type reactor. Further, the supercritical water unit 120 may be horizontal flow, upflow, or downflow. Further yet, the supercritical water unit 120 may have a residence time of from 10 seconds to 60 minutes; an operating temperature of from 400° C. to 650° C. (such that the supercritical water unit 120 operates at supercritical water conditions); or both.

As previously stated, the supercritical water unit 120 may receive the water 6 and the hydrocarbon-containing stream 4. In embodiments, the water 6 and/or hydrocarbon-containing stream 4 may be pre-mixed at a feed mixer (such as where the water 6 and the hydrocarbon-containing stream 4 meet in the figures) prior to being introduced to the supercritical water unit 120. The feed mixer may in turn be fluidly connected downstream of the pretreatment unit 102 and the water storage medium 104, as well as upstream of the supercritical water unit 120.

Additionally, the mixture of the water 6 and the hydrocarbon-containing stream 4 may be preheated at a mixture preheater 118. The mixture preheater 118 may preheat the water-hydrocarbon-containing stream mixture 16 to a temperature of from 380° C. to 650° C. to prior to introducing the water-hydrocarbon-containing stream mixture 16 to the supercritical water unit 120. Accordingly, the mixture preheater 118 may be interposed downstream of the feed mixer and upstream of the supercritical water unit 120.

Further, the system 100 may be configured such that the hydrocarbon-containing stream 4 and the water 6 may be introduced at defined ratios either to the feed mixer or supercritical water unit 120. For example, and in embodiments, a hydrocarbon-containing stream to water ratio (by weight) within the system 100 may be from 1:1 to 1:10 hydrocarbon-containing stream to water, and/or from 1:1 to 1:5 hydrocarbon-containing stream to water by weight.

As previously stated, the water-hydrocarbon-containing stream mixture 16 may be preheated. Additionally or alternatively, the water 6 and hydrocarbon-containing stream 4 may be preheated individually before introduction to the supercritical water unit 120. For example, and in embodiments, the system 100 may further comprise a water preheater 112 and a feed preheater 116. The feed preheater 116 may be fluidly connected downstream of the pretreatment unit 102, as well as upstream of the supercritical water unit 120 and/or the feed mixer. The water preheater 112 may be fluidly connected downstream of the water storage medium 104, as well as upstream of the supercritical water unit 120 and/or the feed mixer. The feed preheater 116 may preheat the hydrocarbon-containing stream 4 to a temperature of from 50° C. to 350° C. prior to being introduced to the supercritical water unit 120 or the feed mixer. Similarly, the water preheater 112 may preheat the water 6 to a temperature of from 374° C. to 800° C. prior to being introduced to the supercritical water unit 120 or the feed mixer.

Still referring to FIG. 1, the water 6 and/or hydrocarbon-containing stream 4 may also be pressurized prior to being introduced to the supercritical water unit 120, the preheaters, or mixers. For example, and in embodiments, the system 100 may comprise a feed pump 106 fluidly connected downstream of the pretreatment unit 102 and upstream of the supercritical water unit 120. Similarly, the system 100 may comprise a water pump 108 fluidly connected downstream of the water storage medium 104 and upstream of the supercritical water unit 120.

Still referring to FIG. 1, and as previously stated, the supercritical water unit 120 may receive the water 6 and the hydrocarbon-containing stream 4 for upgrading the hydrocarbon-containing stream 4 into a linear alpha olefin-containing product 20. However, the system 100 may further include one or more downstream separation units for separating the linear alpha olefins from the remainder of the product. For example, and as shown in FIG. 1, the system 100 may further comprise a gas-liquid separator 124 fluidly connected downstream of the supercritical water unit 120. The gas-liquid separator 124 may be configured to separate the linear alpha olefin-containing product 20 into a gas product stream 22 and a liquid product stream 24.

The system 100 may further comprise an oil-water separator 128 fluidly connected downstream of the supercritical water unit 120 and the gas-liquid separator 124. The oil-water separator 128 may be configured to separate the liquid product stream 24 into a gas hydrocarbon product 26, a liquid hydrocarbon product 28, and a water residue stream 30.

The system 100 may further comprise an oil-gas separator 126 fluidly connected downstream of the supercritical water unit 120 and the gas-liquid separator 124. The oil-gas separator 126 may be configured to separate the gas product stream 22 into additional gas hydrocarbon product 26, additional liquid hydrocarbon product 28, and additional water residue stream 30 after condensing the gas product stream 22, although at different product stream ratios than the oil-water separator 128.

The system 100 may then comprise a distillation column 130 fluidly connected downstream from the oil-water separator 128 and the oil-gas separator 126. The distillation column 130 may be configured to receive the liquid hydrocarbon product 28 from the oil-water separator 128 and the oil-gas separator 126 and to produce a light distillate 32, a middle distillate 36, and a heavy distillate 34. The system 100 may then comprise a LAO extraction unit 132 fluidly connected downstream from the distillation column 130. The LAO extraction unit 132 may comprise a LAO solvent extraction unit, an adsorption unit or both.

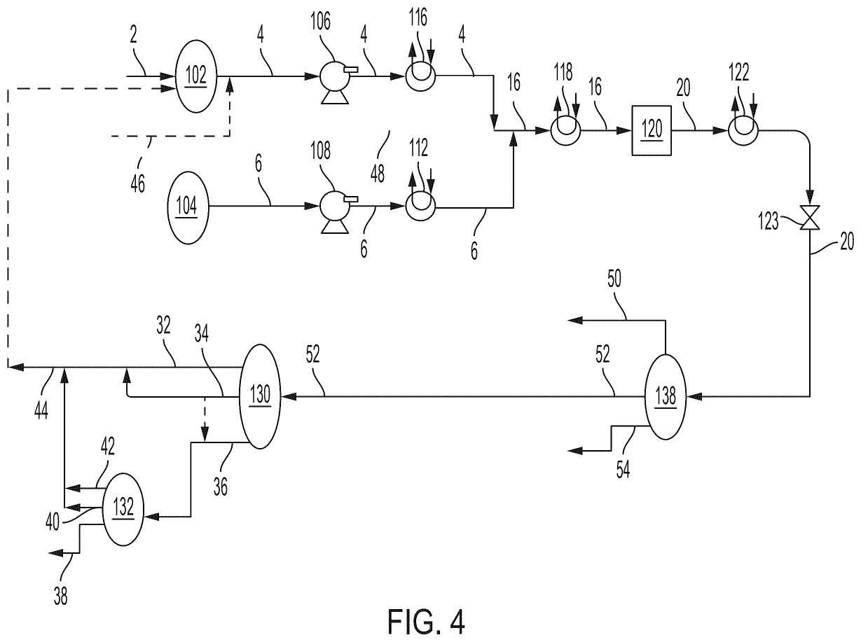

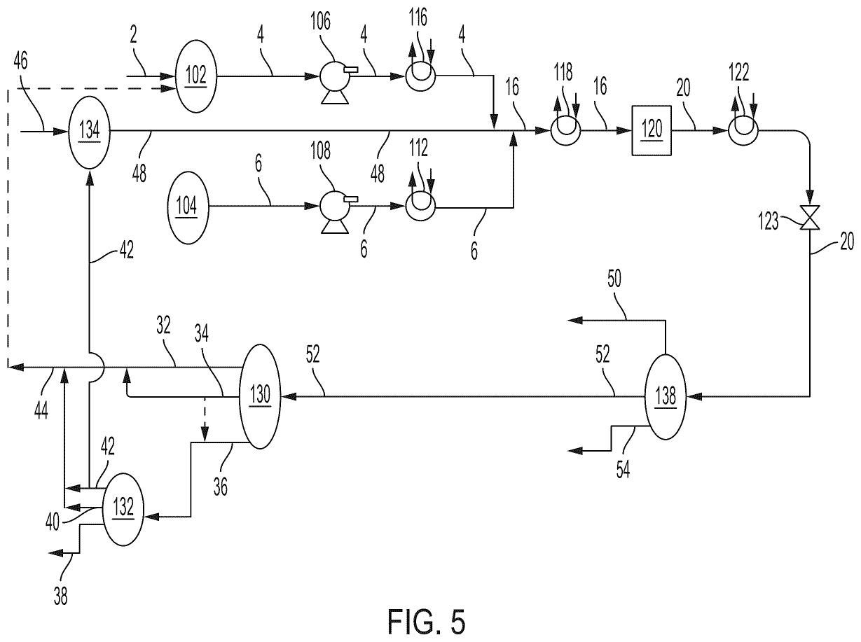

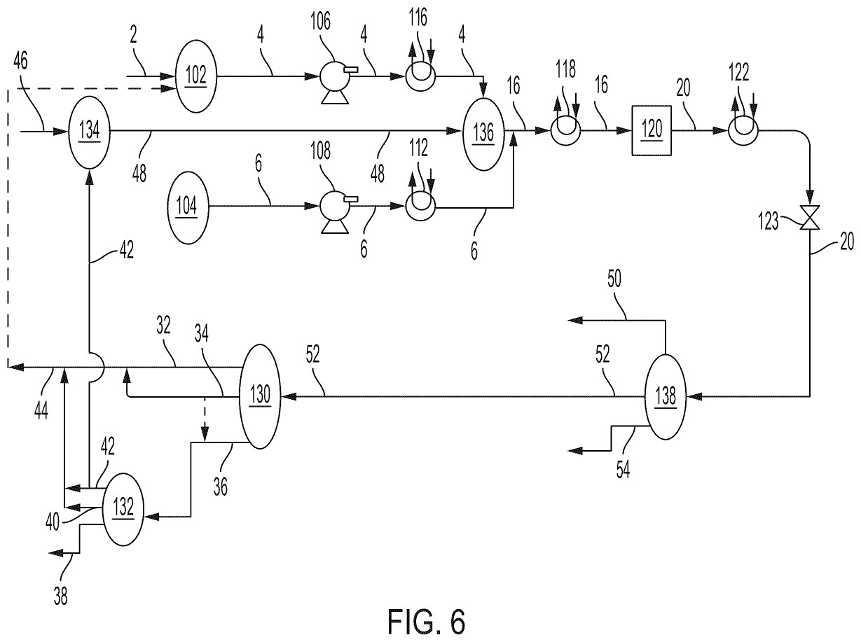

Alternatively, the system 100 may also further comprise a fractionation unit 138, as shown in FIGS. 4-6. The fractionation unit 138 may be fluidly connected downstream from the supercritical water unit 120. The fractionation unit 138 may be configured to separate the linear alpha olefin-containing product 20 into multiple streams by boiling point. Thereby, the products of the fractionation unit 138 may be a gas product 50 having a T₉₅ boiling point of less than 36° C. (i.e. a cutpoint of 36° C.); a liquid distillate product 52 having a T₉₅ boiling point of from 36° C. to 360° C. (i.e. a cutpoint of 360° C.); and a liquid residue product 54 having a T₉₅ boiling point of greater than 360° C., such as from 360° C. to 600° C. (i.e. a remaining liquid residue cut). The fractionation unit 138 may also be configured to form a water product, i.e. the fractionation unit 138 may have an oil-water separation component.

As explained in further detail herein, the linear alpha olefins desired for later polyalphaolefin synthesis may generally have a boiling point between 121° C. (1-Octene) and 213° C. (1-Dodecene), such that the liquid distillate product 52 and/or the liquid hydrocarbon product 28 may be isolated for additional separation of the linear alpha olefins. For example, and still referring to FIG. 1, the system 100 may further comprise the distillation column 130 fluidly connected downstream from the fractionation unit 138, the distillation column 130 configured to receive the liquid distillate product 52 from the fractionation unit 138 or the liquid hydrocarbon product 28 from the gas-liquid separator 124 and oil-water separator 128. The distillation column 130 may be configured to separate the liquid distillate into one or more streams including a light distillate 32 having a T₉₅ boiling point of from 36° C. to 110° C.; a middle distillate 36 having a T₉₅ boiling point of from 110° C. to 220° C.; and a heavy distillate 34 having a T₉₅ boiling point of greater than 220° C., such as from 220° C. to 360° C., from 360° C. to 500° C., from 500° C. to 1000° C., or combinations of the previous ranges or smaller ranges therein.

As previously stated, the linear alpha olefins desired for later polyalphaolefin synthesis may generally have a boiling point between 121° C. and 213° C. Accordingly, to effect these separations from the distillate streams, the system 100 may comprise the LAO extraction unit 132, which may comprise an adsorption unit fluidly connected downstream from the distillation column 130. The adsorption unit may have one or more adsorption beds configured to adsorb a linear alpha olefin from the middle distillate 36, the heavy distillate 34, or both when introduced to the adsorption unit. Particularly, a linear alpha olefin fraction of the middle distillate 36, the heavy distillate 34, or both may be adsorbed onto the one or more adsorption beds of the one or more adsorption units, thereby forming an adsorbed linear olefin fraction, a unadsorbed paraffin fraction 40, and an unadsorbed aromatics fraction 42. The adsorption unit may then be configured to desorb the adsorbed linear alpha olefin fraction from the one or more adsorption beds, such as by depressurization or heating or solvent flushing with an aromatic compound, thereby forming a linear alpha olefin product 38. In embodiments, the one or more adsorption units may be any adsorption unit known in the art, such as, but not limited to, a pressure swing adsorption (PSA) or temperature swing adsorption (TSA) unit.

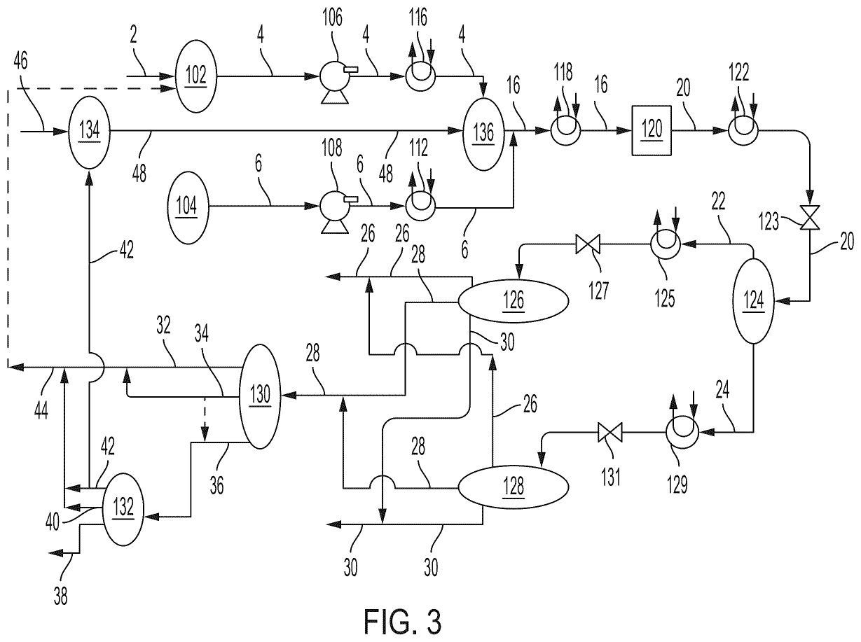

Still referring to FIGS. 1-6, in at least some embodiments, the linear alpha olefin-containing product 20 may be quenched, depressurized or both prior to being sent to the one or more separation units. Accordingly, the system 100 may further comprise a quenching unit 122 and a depressurization unit 123. The quenching unit 122 may be similar or identical to the feed preheater 116 and/or water preheater 112, i.e., the quenching unit may be a heat exchanger configured to cool/quench the linear alpha olefin-containing product 20 to a temperature of from 20° C. to 250° C., such as with a heat exchange fluid such as, but not limited to, water or steam. The depressurization unit 121 may be configured to depressurize the linear alpha olefin-containing product 20 to a pressure of from 0.1 megapascals (MPa) to 5 MPa.

The system may also comprise heat exchangers 125/129 for cooling the gas product stream 22 and the liquid product stream 24 prior to introduction into the oil-gas separator 126 and the oil-liquid separator 128, respectively, such as with a heat exchange fluid such as, but not limited to, water or steam. Similarly, the system may also comprise depressurizers 127/131 for depressurizing the gas product stream 22 and the liquid product stream 24 prior to introduction into the oil-gas separator 126 and the oil-liquid separator 128, respectively.

As previously stated, there may be various heat exchangers, preheaters, and quench units positioned within the system to heat and cool various streams. Accordingly, in at least some embodiments, the feed preheater 116, the water preheater 112, the mixture preheater 118, the quench unit 122, and the heat exchangers 125/129 may be heat integrated, such that hot heat transfer fluid exiting the quenching units and the heat exchangers may eventually be circulated to the preheaters, and vice versa, such that total energy usage of the system is reduced.

As previously stated, the system 100 may be configured such that a hydrocarbon-containing stream 4 meeting certain parameters is introduced to the supercritical water unit 120, such that production of linear alpha olefins is increased. However, in embodiments, supplemental feeds may be introduced with the hydrocarbon-containing stream 4 to take advantage of certain synergies between the components of the primary and supplemental streams.

For example, and as shown in FIGS. 1 and 4, a waste plastics fraction 46 may be introduced with the hydrocarbon-containing stream 4. The waste plastics fraction 46 may comprise polyolefins, such as but not limited to high density polyethylene (HDPE). Without being limited by theory, it is contemplated that introducing the waste plastics fraction 46 may provide a hydrogen donor compound source for hydrogen abstraction reaction mechanisms within the supercritical water unit 120.

Particularly, it is contemplated that after the waste plastics and a non-paraffinic portion (such as asphaltenes) of the hydrocarbon-containing stream 4 undergo initial beta-scission, the resulting radicals from the paraffinic and non-paraffinic fractions may abstract hydrogen from the waste plastics, thereby moving the internal reactions within the caging supercritical water medium from cyclization and/or condensation to hydrogen abstraction. This may in turn suppress formation of coke and aromatics from the paraffinic and non-paraffinic (such as asphaltene) fractions and drive products to olefins.

Further, where the waste plastics fraction 46 comprises primarily HDPE or other low branching polyolefins, yields of olefins such as linear alpha olefins may be further increased. Accordingly, in at least some embodiments, the waste plastics fraction 46 may comprise less than 10 wt. % branched polyolefins, such as LDPE and LLDPE, such as from 0 wt. % to 0.1 wt. %, from 0.1 wt. % to 1 wt. %, from 1 wt. % to 10 wt. %, or combinations of the previous ranges or smaller ranges therein, such as from 0 wt. % to 10 wt. % branched polyolefins.

However, due to the caging effect of supercritical water and the hydrolysis effect of near-critical water, there may be concern that the waste plastic fraction and the hydrocarbon-containing stream 4 need to be adequately distributed prior to exposure to the water 6 medium.

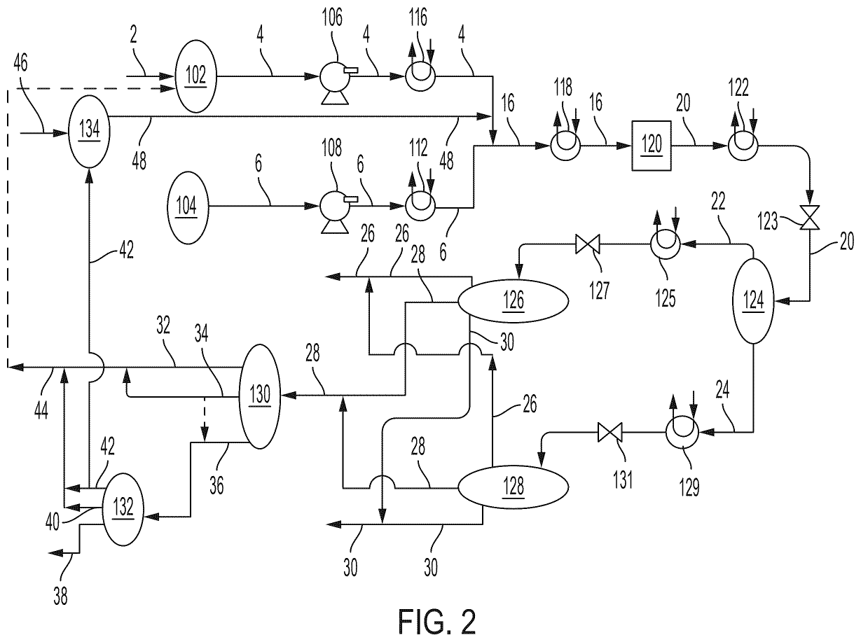

Accordingly, as shown in FIGS. 2 and 4, the system 100 may further comprise a first mixing unit 134 fluidly connected downstream from the LAO extraction unit 132 and upstream from the supercritical water unit 120. The first mixing unit 134 may receive the unadsorbed aromatics fraction 42 as well as the waste plastics fraction 46 and mix the same. In embodiments, the unadsorbed aromatics fraction 42 may comprise benzene, toluene, xylene, alkylated benzenes, naphthalene, alkylated naphthalenes, or combinations thereof. Accordingly, by mixing the unadsorbed aromatics fraction 42 and the waste plastics fraction 46, the unadsorbed aromatics fraction 42 may be taken into a polymer framework of the waste plastics fraction 46, thereby swelling the waste plastics fraction 46 and forming a swollen waste plastics fraction 48. Accordingly, the system 100 may then be configured such that the swollen waste plastics fraction 48 is introduced to the supercritical water unit 120 with the hydrocarbon-containing stream 4.

However, if adequate mixing of the hydrocarbon-containing stream 4 and the swollen waste plastics fraction 48 remains a concern, the system 100 may further comprise a second mixing unit 136 fluidly connected downstream from the pretreatment unit 102 and the first mixing unit 134, and upstream from the supercritical water unit 120. The second mixing unit 136 may be configured to receive the hydrocarbon-containing stream 4 from the pretreatment unit 102 and the swollen waste plastics fraction 48 from the first mixing unit 134 as well as to mix the two prior to the introduction to the supercritical water unit 120.

Without being limited by theory, pre-swelling the swollen waste plastics fraction 48 prior to introduction into the supercritical water may ensure the swollen waste plastics are well distributed with the hydrocarbon-containing stream 4. Particularly, the waste plastics fraction 46 may not only suppress condensation reactions of the paraffinic and non-paraffinic fractions, but additionally suppress condensation reactions with the unabsorbed aromatics fraction. This may be beneficial as aromatic fractions are particularly susceptible to coking through condensation reactions, such that the combination of the waste plastics fraction 46 and the aromatics fraction, especially in the swelled distributed form, may expand the potential feeds useable with the supercritical water unit 120 while still preferentially resulting in linear alpha olefin products.

Referring back to FIG. 1, and as previously stated, the hydrocarbon-containing stream 4 and the water 6 may be preheated and/or pre-mixed prior to introduction to the supercritical water unit 120. Accordingly, the feed mixer, the mixture preheater 118, the water preheater 112, and the feed preheater 116 may be interposed at any position in the fluid linkages between the pretreatment unit 102, the first and second mixers, and the supercritical water unit 120. In one non-limiting embodiment for example, the feed mixer may be positioned downstream of the second mixer and upstream of the supercritical water unit 120, with the mixer preheater being positioned downstream of the feed mixer.

As previously stated, embodiments herein may also be directed to processes of producing linear alpha olefins utilizing the supercritical water unit 120. The methods may include any of the systems hereinbefore described.

In at least some embodiments, the process may comprise introducing the hydrocarbon-containing stream 4 and the water 6 to the supercritical water unit 120; and upgrading the hydrocarbon-containing stream 4 with the water 6 within the supercritical water unit 120, thereby forming a linear alpha olefin-containing product 20, and wherein the water 6 is either at supercritical conditions prior to entering the supercritical water unit 120 or converted to supercritical conditions within the supercritical water unit 120. The process may also comprise pretreating the carbon-containing stream 2 in the pretreatment unit 102 to thereby form the hydrocarbon-containing stream 4.

As previously stated, the linear alpha olefin-containing product 20 may be separated in one or more units to isolate the linear alpha olefins. Accordingly, the process may further comprise introducing the linear alpha olefin-containing product 20 to the fractionation unit 138, thereby forming the gas product 50, the liquid distillate product 52, and the liquid residue product 54. The process may also comprise introducing the liquid distillate product 52 to the distillation column 130, thereby forming the light distillate 32, the middle distillate 36, and the heavy distillate 34. In at least some embodiments, the fractionation unit 138 may also have an oil-water separation function, such that a water stream (not illustrated) may also be formed.

Alternatively, the process may further comprise introducing the linear alpha olefin-containing product 20 to the gas-liquid separator 124 to form the gas product stream 22 and the liquid product stream 24. The process may then comprise introducing the gas product stream 22 to the oil-gas separator 126 and the liquid product stream 24 to the oil-water separator 128 to form the gas hydrocarbon product 26, the liquid hydrocarbon product 28, and the water residue stream 30. The process may then comprise introducing the liquid hydrocarbon product 28 to the distillation column 130 to form the light distillate 32, the middle distillate 36, and the heavy distillate 34.

The process may also comprise introducing the middle distillate 36, the heavy distillate 34, or both to the one or more adsorption units; adsorbing a linear alpha olefin fraction of the middle distillate 36 fraction, the heavy distillate 34 fraction, or both onto one or more adsorption beds of the one or more adsorption units, thereby forming an adsorbed linear olefin fraction, a unabsorbed paraffin fraction, and an unabsorbed aromatic fraction; desorbing the adsorbed linear alpha olefin fraction from the one or more adsorption beds, thereby forming a linear alpha olefin product 38; or combinations thereof.

As previously stated, it may be desired in at least some embodiments to pretreat the hydrocarbon-containing stream 4 such that sulfur content is reduced. Accordingly, the process may comprise pretreating a carbon-containing stream 2 in a hydroprocessor selected from a hydrocracker, a hydrotreater, or both to form the hydrocarbon-containing stream 4.

As previously stated, it may also be desired in at least some embodiments to recycle non-LAO product streams back to the pretreatment unit and/or supercritical water unit for formation of additional hydrocarbon-containing stream and/or linear alpha olefin-containing product stream. Accordingly, the process may also comprise recycling the unabsorbed aromatics fraction, the light distillate stream, the middle distillate stream, the heavy distillate stream, or combinations thereof to the hydroprocessor with the carbon-containing stream 2 to form additional hydrocarbon-containing stream 4. The process may also comprise recycling the unabsorbed paraffin fraction as additional hydrocarbon-containing stream 4 to the supercritical water unit 120.

As previously stated, it may be desired to introduce a waste plastics stream or a swollen waste plastics stream to the hydrocarbon-containing stream 4. Accordingly, the process may comprise introducing the waste plastics fraction 46 into the supercritical water unit 120 along with the hydrocarbon-containing stream 4 and the water 6, thereby forming additional linear alpha olefin-containing product 20. The process may also comprise mixing the unabsorbed aromatics fraction from the one or more adsorption units with the waste plastics fraction 46, thereby forming a swollen waste plastics stream; and introducing the swollen waste plastics stream to the supercritical water unit 120 along with the hydrocarbon-containing stream 4 and the water 6, thereby forming additional linear alpha olefin-containing product 20. Further, the process may comprise mixing the swollen waste plastics stream and the hydrocarbon-containing stream 4 prior to introducing the swollen waste plastics stream and the hydrocarbon-containing stream 4 to the supercritical water unit 120.

Also as previously stated, it may be desired to preheat and/or mix the hydrocarbon-containing stream 4 and the water 6 prior to introduction to the supercritical water unit 120. Accordingly, the process may comprise mixing the water 6 and the hydrocarbon-containing stream 4 prior to the water 6 and the hydrocarbon-containing stream 4 being introduced to the supercritical water unit 120; and preheating the water-hydrocarbon-containing stream mixture 16 to a temperature of from 380° C. to 650° C. prior to introducing the water-hydrocarbon-containing stream mixture 16 to the supercritical water unit 120.

### EXAMPLES

One or more simulations were conducted to determine the suitability of the systems and feeds described herein for upgrading to linear alpha olefins. Example 1 utilized an unconverted oil (UCO) from a hydroprocessing unit, specifically a hydrocracker, for the hydrocarbon-containing stream 4. The feed to the hydroprocessing unit was vacuum gas oil range hydrocarbons. The properties of the unconverted oil and the mass flow results are shown in Tables 1 and 2, respectively, below.

TABLE 1
Example 1 Supercritical Unit Feed (UCO) Properties

| | |
|---|---|
| API Gravity [degree] | 37.1 |
| Sulfur [ppm] | 10 |
| Nitrogen Content [ppm] | 0.2 |
| Pour Point [°C] | 43 |
| Kinematic Viscosity @ 100 °C [cSt] | 5.9 |
| Wax Content [wt %] | 28 |
| Aromatics by Volume [%] | 6.5 |
| T₅ Boiling Point [°C] | 392 |
| T₁₀ Boiling Point [°C] | 407 |
| T₃₀ Boiling Point [°C] | 433 |
| T₅₀ Boiling Point [°C] | 457 |
| T₇₀ Boiling Point [°C] | 492 |
| T₉₀ Boiling Point [°C] | 514 |
| T₉₅ Boiling Point [°C] | 541 |

TABLE 2
Mass Flows for Example 1 (UCO)

| | | Temperature | Pressure | Mass Flow |
|---|---|---|---|---|
| Stream # | Stream Description | (°C) | (bar gauge) | (kg/h) |
| 6 | Water | 25 | 5 | 66111 |
| 4 | UCO Feed | 120 | 10 | 55740 |
| 10 | Compressed Water | 124 | 270 | 55740 |
| 8 | Compressed UCO | 27 | 270 | 66111 |
| 14 | Preheated Water | 250 | 270 | 55740 |
| 12 | Preheated UCO | 525 | 270 | 66111 |
| 16 | Water-UCO Mixture | 419 | 270 | 121851 |
| 18 | Preheated Feed Mixture | 470 | 270 | 121851 |
| 20 | LAO-Containing Product | 470 | 270 | 121851 |
| 22 | Gas Product Stream | 195 | 18 | 20203 |
| 24 | Liquid Product Stream | 195 | 18 | 101648 |
| 26 | Gas Hydrocarbon Product Stream | 90 | 1 | 36 |
| | (oil-water separator) | | | |
| 28 | Liquid hydrocarbon product | 90 | 1 | 43696 |
| | (oil-water separator) | | | |
| 30 | Water Residue Stream | 90 | 1 | 57915 |
| | (oil-water separator) | | | |
| 26 | Gas Hydrocarbon Product Stream | 37 | 1 | 2812 |
| | (gas-oil separator) | | | |
| 28 | Liquid hydrocarbon product | 37 | 1 | 9223 |
| | (gas-oil separator) | | | |
| 30 | Water Residue Stream | 37 | 1 | 8168 |
| | (gas-oil separator) | | | |
| 26 | Gas Hydrocarbon Product Stream | 39 | 1 | 2848 |
| | (combined) | | | |
| 28 | Liquid hydrocarbon product (combined) | 80 | 1 | 52919 |
| 30 | Water Residue Stream (combined) | 84 | 1 | 66084 |
| 32 | Light Distillate (Very Light) | 35 | 0 | 1929 |
| 32 | Light Distillate (Light) | 35 | 0 | 4513 |
| 36 | Middle Distillate | 35 | 0 | 14338 |
| 34 | Heavy Distillate (Light) | 35 | 0 | 18347 |
| 34 | Heavy Distillate (Heavy) | 35 | 0 | 13768 |
| 38 | LAO Product Stream | 90 | 0 | 7836 |
| 40/42 | Un-adsorbed Product Stream | 90 | 0 | 6502 |
| | (Paraffin and Aromatics) | | | |
| 44 | Potential Hydroprocessor Returns | 37 | 0 | 45059 |

| | |
|---|---|
| LAO Product Stream Yield | 14.1% |

As shown in Table 1, utilizing a Unconverted Oil from hydrocracking resulted in an LAO conversion percentage of above 14 percent. For comparison with a higher sulfur alternative, Example 2 utilized a deasphalted oil. The properties of the deasphalted oil and the mass flow results are shown in Tables 3 and 4, respectively, below.

TABLE 3
Example 2 Supercritical Unit Feed (Deasphalted Oil) Properties

| | |
|---|---|
| API Gravity [degree] | 20.6 |
| Sulfur [ppm] | 21,400 |
| Nitrogen Content [ppm] | 737 |
| Pour Point [°C] | 72 |
| Kinematic Viscosity @ 100 °C [cSt] | 137 |
| Aromatics by Volume [%] | 32 |
| T₅ Boiling Point [°C] | 473 |
| T₁₀ Boiling Point [°C] | 497 |
| T₃₀ Boiling Point [°C] | 546 |
| T₅₀ Boiling Point [°C] | 573 |
| T₇₀ Boiling Point [°C] | 601 |
| T₉₀ Boiling Point [°C] | 649 |
| T₉₅ Boiling Point [°C] | 673 |

TABLE 4
Mass Flows for Example 1 (UCO)

| | | | Pressure | Mass |
|---|---|---|---|---|
| Stream | | Temperature | (bar | Flow |
| # | Stream Description | (°C) | gauge) | (kg/h) |
| 6 | Water | 25 | 5 | 66111 |
| 4 | UCO Feed | 120 | 10 | 61586 |
| 10 | Compressed Water | 122 | 270 | 61586 |
| 8 | Compressed UCO | 27 | 270 | 66111 |
| 14 | Preheated Water | 244 | 270 | 61586 |
| 12 | Preheated UCO | 531 | 270 | 66111 |
| 16 | Water-UCO Mixture | 419 | 270 | 127697 |
| 18 | Preheated Feed Mixture | 465 | 270 | 127697 |
| 20 | LAO-Containing Product | 465 | 270 | 127697 |
| 22 | Gas Product Stream | 200 | 18 | 15383 |
| 24 | Liquid Product Stream | 200 | 18 | 112314 |
| 26 | Gas Hydrocarbon Product Stream | 91 | 1 | 9 |
| | (oil-water separator) | | | |
| 28 | Liquid hydrocarbon product | 91 | 1 | 54436 |
| | (oil-water separator) | | | |
| 30 | Water Residue Stream | 91 | 1 | 57869 |
| | (oil-water separator) | | | |
| 26 | Gas Hydrocarbon Product Stream | 37 | 1 | 2554 |
| | (gas-oil separator) | | | |
| 28 | Liquid hydrocarbon product | 37 | 1 | 4617 |
| | (gas-oil separator) | | | |
| 30 | Water Residue Stream | 37 | 1 | 8212 |
| | (gas-oil separator) | | | |
| 26 | Gas Hydrocarbon Product | 38 | 1 | 2563 |
| | Stream (combined) | | | |
| 28 | Liquid hydrocarbon product | 86 | 1 | 59053 |
| | (combined) | | | |
| 30 | Water Residue Stream (combined) | 84 | 1 | 66082 |
| 32 | Light Distillate (Very Light) | 17 | 0 | 852 |
| 32 | Light Distillate (Light) | 56 | 0 | 1605 |
| 36 | Middle Distillate (feed to | 156 | 0 | 7955 |
| | LAO extractor) | | | |
| 34 | Heavy Distillate (Light) | 260 | 0 | 12860 |
| 34 | Heavy Distillate (Heavy) | 456 | 0 | 35760 |
| 38 | LAO Product Stream | 90 | 0 | 4320 |
| 40/42 | Un-adsorbed Product Stream | 173 | 0 | 3635 |
| | (Paraffin and Aromatics) | | | |
| 44 | Potential Hydroprocessor Returns | 361 | 0 | 54713 |

| | |
|---|---|
| LAO Product Stream Yield | 7.0% |

As shown in Table 2 as compared to Table 4, the lower sulfur UCO had over double the ultimate LAO product stream yield than that of the high sulfur deasphalted oil, illustrating the importance of feed selection and sulfur content in the yields of supercritical water hydrocarbon upgrading for LAOs. Further, the amount of middle distillate, and thus the amount of LAO, available within the system was also reduced for Example 2 (7955) as compared to Example 1 (14,338).

Noticeably however, the deasphalted oil of Example 2 actually showed a slightly more favorable profile of C₈-C₁₂ LAOs than that of the UCO of Example 1, as shown in Table 5 below.

TABLE 5
LAO Distribution Comparison of Examples 1 and 2

| | Example 1 | Example 2 |
|---|---|---|
| Mass Fraction | Middle Distillate | Middle Distillate |
| 1-Pentene | 0.0% | 0.0% |
| 1-Hexene | 0.3% | 0.1% |
| 1-Heptene | 1.8% | 0.7% |
| 1-Octene | 6.4% | 3.6% |
| 1-Nonene | 13.3% | 9.6% |
| 1-Decene | 17.1% | 16.7% |
| 1-Undecene | 11.4% | 14.7% |
| 1-Dodecene | 5.8% | 12.8% |
| 1-Tridecene | 1.7% | 4.6% |
| 1-Tetradecen | 0.2% | 0.9% |
| 1-Pentadecen | 0.0% | 0.1% |
| 1-Hexadecene | 0.0% | 0.0% |
| 1-Heptadecen | 0.0% | 0.0% |
| 1-Octadecene | 0.0% | 0.0% |
| 1-Nonadecene | 0.0% | 0.0% |
| 1-Eicosene | 0.0% | 0.0% |
| 1-C21═ | 0.0% | 0.0% |
| 1-C22═ | 0.0% | 0.0% |
| 1-C23═ | 0.0% | 0.0% |
| 1-C24═ | 0.0% | 0.0% |
| C8-C12 LAO Fractions | 54.0% | 57.3% |
| Apparent Percentage of | 54.6% | 54.3% |
| Middle Distillate produced | | |
| as LAO product stream | | |
| Actual LAO Recovery From | 94% | 55% |
| Middle Distillate in LAO | | |
| Extractor | | |

However, this improved profile was more than offset by difficulties in adequately extracting the LAOs from the middle distillation stream. Particularly, as shown in Table 5 above, while there were similar recovery yield for the LAO product streams in Examples 1 and 2 as a percentage of mass flow to the LAO extractor, the actual LAO concentration in the streams greatly varied.

Without being limited by theory, this may be attributable to the competition for adsorption sites between LAOs and sulfur, such that the increased sulfur content in Example 2 reduced the total yield of LAOs in favor of greater amounts of sulfur and sulfur-containing hydrocarbons. This is particularly frustrating as to LAO extraction, as the normal means for desulfurization, hydrotreating, will also saturate the stream and thus remove LAOs.

Accordingly, these Examples illustrate that the inclusion of sulfur compounds and thereby sulfur radicals may be beneficial to some extent in LAO generation, but also negative to a remaining extent to LAO yield and/or extraction. Accordingly, by balancing the amount of sulfur within the supercritical water unit feed as in embodiments herein, LAO yield and subsequent extraction can be maximized.

It is noted that recitations in the present disclosure of a component of the present disclosure being “configured”, “operable”, or “sufficient” in a particular way, to embody a particular property, or to function in a particular manner, are structural recitations, as opposed to recitations of intended use. More specifically, the references in the present disclosure to the manner in which a component is “configured, “operable”, or “sufficient” denotes an existing physical condition of the component and, as such, is to be taken as a definite recitation of the structural characteristics of the component.

It is also noted that recitations herein of “at least one” component, element, etc., should not be used to create an inference that the alternative use of the articles “a” or “an” should be limited to a single component, element, etc. The singular forms “a,” “an” and “the” include plural referents, unless the context clearly dictates otherwise.

Throughout this disclosure ranges are provided. It is envisioned that each discrete value encompassed by the ranges are also included. Additionally, the ranges which may be formed by each discrete value encompassed by the explicitly disclosed ranges are equally envisioned.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.” It is noted that the use of the terms “having” or “including”, or grammatical variations thereof, in this disclosure should also be interpreted in like manner as the more commonly used open-ended preamble term “comprising”.

As used in this disclosure, terms such as “first” and “second” are arbitrarily assigned and are merely intended to differentiate between two or more instances or components. It is to be understood that the words “first” and “second” serve no other purpose and are not part of the name or description of the component, nor do they necessarily define a relative location, position, or order of the component. Furthermore, it is to be understood that the mere use of the term “first” and “second” does not require that there be any “third” component, although that possibility is contemplated under the scope of the present disclosure.

It is noted that terms like “preferably,” “commonly,” and “typically,” when utilized herein, are not utilized to limit the scope of the claimed invention or to imply that certain features are critical, essential, or even important to the structure or function of the claimed invention. Rather, these terms are merely intended to identify particular aspects of an embodiment of the present disclosure or to emphasize alternative or additional features that may or may not be utilized in a particular embodiment of the present disclosure.

Having described the subject matter of the present embodiments herein in detail and by reference to specific embodiments thereof, it is noted that the various details disclosed herein should not be taken to imply that these details relate to elements that are essential components of the various embodiments described herein, even in cases where a particular element is illustrated in each of the drawings that accompany the present description. Further, it will be apparent that modifications and variations are possible without departing from the scope of the present embodiments including, but not limited to, embodiments defined in the appended claims. More specifically, although some aspects of the present embodiments are identified herein as preferred or particularly advantageous, it is contemplated that the present embodiments is not necessarily limited to these aspects.

1. A process for producing linear alpha olefins utilizing a supercritical water unit, the process comprising:
introducing a hydrocarbon-containing stream and water to a supercritical water unit, wherein the water is either at supercritical conditions prior to entering the supercritical water unit or converted to supercritical conditions within the supercritical water unit; and
upgrading the hydrocarbon-containing stream with the water within the supercritical water unit, thereby forming a linear alpha olefin-containing product, and wherein:
the hydrocarbon-containing stream comprises greater than or equal to 90 vol. % paraffins,
the hydrocarbon-containing stream comprises from 0.1 vol. % to 10 vol. % aromatics, and
the hydrocarbon-containing stream comprises from 0.1 ppm to 100 ppm sulfur.

2. The process of claim 1, further comprising pretreating a carbon-containing stream in a pretreatment unit to thereby form the hydrocarbon-containing stream, the carbon-containing stream comprising carbon dioxide, carbon monoxide, alcohol, crude oil, plastics, biomass, coal, or combinations thereof.

3. The process of claim 2, wherein the carbon-containing stream comprises a vacuum gas oil, a vacuum residue fraction, or combinations thereof.

4. The process of claim 2, wherein:
the pretreatment unit comprises a hydroprocessor; and
the hydrocarbon-containing stream is:
a hydroprocessor bottoms residue fraction,
a hydroprocessor unconverted oil fraction, or
both.

5. The process of claim 4, wherein the pretreatment unit further comprises the solvent extraction unit upstream of the hydroprocessor.

6. The process of claim 1, further comprising:
introducing the linear alpha olefin-containing product to a gas-liquid separator to form a gas product stream and a liquid product stream;
introducing the gas product stream to an oil-gas separator to form a gas hydrocarbon product, a liquid hydrocarbon product, and a residue water stream; and
introducing the liquid product stream to an oil-water separator to form additional gas hydrocarbon product, additional liquid hydrocarbon product, and additional residue water stream.

7. The process of claim 6, further comprising introducing the liquid hydrocarbon product to a distillation column, thereby forming:
a light distillate having a T₉₅ boiling point of from 36° C. to 110° C.;
a middle distillate having a T₉₅ boiling point of from 110° C. to 220° C.; and
a heavy distillate having a T₉₅ boiling point of from 220° C. to 360° C.

8. The process of claim 1, further comprising introducing the linear alpha olefin-containing product to a fractionation unit, thereby forming:
a gas product having a T₉₅ boiling point of less than 36° C.;
a liquid distillate product having a T₉₅ boiling point of from 36° C. to 360° C.; and
a liquid residue product having a T₉₅ boiling point of greater than 360° C.

9. The process of claim 8, further comprising introducing the liquid distillate product to a distillation column, thereby forming:
a light distillate having a T₉₅ boiling point of from 36° C. to 110° C.;
a middle distillate having a T₉₅ boiling point of from 110° C. to 220° C.; and
a heavy distillate having a T₉₅ boiling point of from 220° C. to 360° C.

10. The process of claim 9, further comprising:
introducing the middle distillate, the heavy distillate, or both to an adsorption unit;
adsorbing a linear alpha olefin fraction of the middle distillate, the heavy distillate, or both onto one or more adsorption beds of the adsorption unit, thereby forming an adsorbed linear olefin fraction, a unabsorbed paraffin fraction, and an unabsorbed aromatic fraction; and
desorbing the adsorbed linear alpha olefin fraction from the one or more adsorption beds, thereby forming a linear alpha olefin product.

11. The process of claim 10, further comprising:
pretreating a carbon-containing stream in a hydroprocessor selected from a hydrocracker, a hydrotreater, or both to form the hydrocarbon-containing stream;
recycling a portion of the middle distillate not sent to the adsorption unit, a portion of the heavy distillate not sent to the adsorption unit, the light distillate, the unabsorbed aromatics fraction, or combinations thereof to the hydroprocessor with the carbon-containing stream to form additional hydrocarbon-containing stream; and
recycling the unabsorbed paraffin fraction as additional hydrocarbon-containing stream to the supercritical water unit.

12. The process of claim 11, wherein the carbon-containing stream comprises a vacuum gas oil, a vacuum residue fraction, or combinations thereof.

13. The process of claim 1, further comprising introducing a waste plastics fraction comprising high density polyethylene (HDPE) to the supercritical water upgrading unit along with the hydrocarbon-containing stream and the water, thereby forming additional linear alpha olefin-containing product, and wherein:
the hydrocarbon-containing stream further comprises asphaltenes.

14. The process of claim 13, wherein the waste plastic fraction comprises less than 10 wt. % branched polyolefins.

15. The process of claim 10, further comprising:
mixing the unabsorbed aromatics fraction from the adsorption unit with a waste plastics fraction comprising high density polyethylene (HDPE), thereby forming a swollen waste plastics stream; and
introducing the swollen waste plastics stream to the supercritical water upgrading unit along with the hydrocarbon-containing stream and the water, thereby forming additional linear alpha olefin-containing product, and wherein:
the hydrocarbon-containing stream further comprises asphaltenes and from 0.1 ppm to 100 ppm sulfur.

16. The process of claim 15, further comprising mixing the swollen waste plastics stream and the hydrocarbon-containing stream prior to introducing the swollen waste plastics stream and the hydrocarbon-containing stream to the supercritical water unit.

17. The process of claim 1, wherein the hydrocarbon-containing stream has:
less than 10 ppm nitrogen;
less than 100 ppm oxygen;
a T₉₅ boiling point of from 174° C. to 600° C.;
over 95 vol. % C₁₀-C₄₆ fractions; or
combinations thereof.

18. The process of claim 1, wherein:
a hydrocarbon-containing stream to water ratio introduced to the supercritical water unit is from 10:1 to 1:10 hydrocarbon-containing stream to water;
the water is preheated to a temperature of from 374° C. to 800° C. prior to being introduced to the supercritical water unit; and
the hydrocarbon-containing stream is preheated to a temperature of from 50° C. to 350° C. prior to being introduced to the supercritical water unit.

19. The process of claim 1, further comprising:
mixing the water and the hydrocarbon-containing stream prior to the water and the hydrocarbon-containing stream being introduced to the supercritical water unit; and
preheating the water-hydrocarbon-containing stream mixture to a temperature of from 380° C. to 650° C. prior to introducing the water-hydrocarbon-containing stream mixture to the supercritical water unit.

20. A system for producing linear alpha olefins, the system comprising:
a pre-treatment unit configured to receive a carbon-containing stream;
a supercritical water unit, wherein:
the supercritical water unit is fluidly connected downstream from the pre-treatment unit,
the supercritical water unit is configured to receive water and a hydrocarbon-containing stream-swollen waste plastic stream mixture from a second mixing unit, and
the supercritical water unit is configured to operate under supercritical water conditions;
one or more separation units fluidly connected downstream of the supercritical water unit, the one or more separation units configured to receive a linear alpha olefin-containing product from the supercritical water unit
a distillation column fluidly connected downstream of the supercritical water unit and the one or more separation units, the distillation column configured to receive a liquid distillate product or a liquid hydrocarbon stream from the one or more separation units;
an adsorption unit fluidly connected downstream of the supercritical water unit, the adsorption unit configured to receive a middle distillate, a heavy distillate, or both from the distillation column and produce at least a linear alpha olefin product;
a first mixing unit fluidly connected downstream of the adsorption unit and configured to receive an unadsorbed aromatics fraction from the adsorption unit and a waste plastics stream; and
the second mixing unit fluidly connected downstream of the first mixing unit and the pre-treatment unit as well as upstream from the supercritical water unit, the second mixing unit configured to receive a hydrocarbon-containing stream from the pre-treatment unit and a swollen waste plastics stream from the first mixing unit.
